(19) United States
(12) Patent Application Publication
(10) Pub. No.: US 2001/0056582 A1
(43) Pub. Date: Dec. 27, 2001

(54) PAIRED MUTATIONS

(76) Inventors: MARK BODMER, CAMBRIDGE (GB); JAMIE WILLIAM FOSTER, CAMBRIDGE (GB); ANDREW SANDHAM, CAMBRIDGE (GB); ALAN J. SCHAFER, CAMBRIDGE (GB)

Correspondence Address: MICHAEL C CERRONE, LEGAL DEPARTMENT, INCYTE GENOMICS INC, 3160 PORTER DRIVE, PALO ALTO, CA, 94304, US

(21) Appl. No.: 09/108,277

(22) Filed: Jul. 1, 1998

Related U.S. Application Data
(60) Provisional application No. 60/051,560, filed on Jul. 2, 1997.

(51) Int. Cl. A01K 67/00; C12N 5/06; A61K 48/00; C07H 21/04

(52) U.S. Cl. 800/21; 514/44; 435/325; 435/455; 536/23.1

(57) ABSTRACT

The invention provides a collection of paired samples from a plurality of mutagenized animals, wherein each paired sample comprises: a first sample comprising genetic screening material of an animal; and a second sample comprising reproductive material of that same animal. A sample of reproductive material according to the invention can be used to generate a progeny animal carrying that same mutation. The invention also provides a collection of pairs of mutant genomes, comprising a plurality of animals carrying induced mutations and a corresponding plurality of sample genomic material, wherein a pair of mutated genomes of the collection comprises an animal having a genome containing an induced mutation and the animal's sample genomic material. The mutant animal, which might need to be bred to homozygosity, is useful for investigating the function of the gene. It might also be a useful animal model for studying human disease.

### BACKGROUND OF THE INVENTION

This invention relates to collections of biological samples useful for identifying members of an animal population carrying mutations in a gene of interest.

It is an object of the invention to provide a collection of paired mutations in which genetic information for genome analysis is preserved and paired such that the collection advantageously provides for the ability to produce progeny for genotype and/or phenotype analysis.

### SUMMARY OF THE INVENTION

The invention provides a collection of paired samples from a plurality of mutagenized animals, wherein each of the paired samples comprises: a first sample comprising genetic screening material of a parent animal; and a second sample comprising reproductive material of that same parent animal.

There is further provided a process for producing such a collection, comprising the steps of obtaining pairs of samples from a plurality of mutagenized animals: a first sample comprising genetic screening material; and a second sample comprising reproductive material. The animals may be sacrificed before, during, or after the samples are obtained. The process preferably includes the step of mutagenizing a plurality of animals prior to obtaining the samples.

Preferably, the plurality comprises 100 or more mutagenized animals, more preferably 1000 or more animals, more preferably again 10,000 or more animals, and most preferably at least 100,000 animals, and even up to 1 million animals. Preferably the animals are all of the same species.

The term “mutagenized” means that the animals from which the samples are obtained carry mutations at a frequency characteristic of exposure to mutagenizing conditions. The term “mutagenized” does not refer to spontaneous or background mutations, which are characterized by their low frequency of occurrence, typically one mutation per 50,000-80,000 animals. Therefore, “mutagenizing conditions” refers to artificially (i.e., laboratory) induced mutagenesis, that is performed in vivo, i.e., directly to the animal. The animals from which the samples are obtained preferably carry mutations, determined as phenotypic mutations, at a frequency substantially above this low background frequency, that is, greater than 1 per 50,000 animals in any given gene.

The mutations in the samples of genetic screening material and reproductive material must correspond in the sense that they are identical in position in the genome and in the nature of the mutation.

As used herein, “mutation” refers to an alteration in the nucleotide sequence of a given gene or regulatory sequence from the naturally occurring or normal nucleotide sequence. A mutation may be a single nucleotide alteration (deletion, insertion, substitution), or a deletion or substitution of a number of nucleotides, or a chromosomal rearrangement

As used herein, the term “animal” can be of any type, vertebrate or invertebrate, but is preferably a vertebrate. Preferably the vertebrate is a mammal or a fish. Suitable mammals include primates, rodents, lagomorphs, guinea pigs, horses, sheep, cattle, goats, pigs, cats, and dogs. Preferred mammals are mice and rats, while preferred fish are zebrafish and medaka fish.

As used herein, “genetic screening material” in the first sample in each of the paired samples may be any material suitable for genetic analysis. It may be in a form which is itself suitable or it may be in a form from which suitable material can be derived. Accordingly, this includes the animal itself (which contains its own genetic screening material as represented in any one of a number of its tissues), intact cells and cellular extracts from which DNA or RNA may be isolated for use in assays, and purified DNA or RNA, for example, genomic DNA or cDNA. Where DNA, the DNA may be obtained or purified from a diploid cell of an organism. The genetic screening material may be derived from living or dead organisms. The genetic screening material need not be in the same form in each of the paired samples in the collection. For example, it might comprise intact cells in some paired samples and purified DNA in others.

As used herein, “reproductive material” in the second sample in each of the paired samples may be any material which can be used to generate progeny of the parent animal, and thus includes the animal itself (which contains its own reproductive material in the form of its sperm or ova), or more preferably gametes (ie. spermatozoa or ova), or alternatively gametogenic stem cells from which gametes of the parent animal can be produced, an embryo generated using the parent animal's gametes, or embryonic stem cells from such an embryo, cells suitable for nuclear transfer for cloning the parent animal, or nuclei from such cells. The reproductive material need not be in the same form in each of the paired samples in the collection. For example, it might comprise spermatozoa in some paired samples and ova in others.

The two samples in each of the paired samples in the collection might, in practice, be identical. For instance, gametes can be used as genetic screening material and as reproductive material. However, the process of screening the samples of genetic screening material will destroy the viability of the gametes, whereas the gametes which make up the reproductive material must remain viable in order to fertilize other gametes and generate progeny. It is clear, therefore, that where the two components are identical they do, in fact, have different functions.

Furthermore, where the process of screening a collection depletes the supply of genetic screening material and gametes are used for both samples in a pair, the process of screening the collection will also deplete the available reproductive material. Thus, the genetic screening material preferably does not comprise gametes but comprises somatic tissue which is abundantly available.

Indeed, this emphasizes the advantages of using paired samples. Each of the paired samples contains two representations of a mutagenized animal's genetic constitution. Readily available material is used for screening the population for mutants.

The invention encompasses a collection of pairs of mutated genomes comprising a plurality of animals carrying induced mutations and a corresponding plurality of sample genomic material, wherein a said pair of mutated genomes of said collection comprises an animal having a genome containing an induced mutation and the animal's sample genomic material.

Preferably, the animal's sample genomic material is one of reproductive genomic material, such as gametes, for example, sperm or ova, or such as the corresponding isolated DNA, or somatic genomic material, for example, somatic cells or DNA isolated from such cells.

The gametes of an animal which has been exposed to mutagenic conditions may be used directly as a source of both genetic screening material and reproductive material. Alternatively, the animal may be bred in order to obtain progeny (F1, F2, F3 generation etc.) whose mutations are consistent throughout its gametes and somatic tissue. For instance, the animals from which samples are obtained might be the progeny of an inbred (and therefore genetically identical) population which has been mutagenized. For instance, the germline of an animal may be mutagenized, preferably the male germline, and suitable samples can be obtained from its F1 generation progeny.

Alternatively, although it is possible, it is not necessary according to the invention to screen a living population and then breed from individuals in the population, since each sample of genetic screening material is paired with a sample of reproductive material which can be used to generate an animal carrying the same mutation as was identified during screening. In effect, therefore, this aspect of the invention confers the advantage that the mutations in a population of mutagenized animals are immortalized and animals carrying particular mutations of interest can be produced easily without the need to maintain a living animal population.

Preferably, in the collection of paired mutated genomes the mutagen used to induce mutation in the animal is a chemical mutagen, for example an alykylating agent such as ENU.

Preferably, the collection of paired mutated genomes includes animals wherein each animal of said pair carries a mutation at a frequency in the range of 1 mutant copy of a gene per 500-1150,000 animals, more preferably 1/5,000-1/10,000, and most preferably on the order of 1/1,000.

The invention also encompasses a method of generating a collection of paired mutated genomes, comprising mutagenizing reproductive material of a parent animal, breeding the parent animal with an unmutagenized parental mate to produce a plurality of F1 offspring carrying induced mutations, and obtaining a corresponding plurality of sample genomic material of said F1 offspring.

As used herein, the term “genome” refers to nuclear genetic material (DNA) which is contained in a haploid or diploid set of chromosomes.

It is anticipated that the collections according to the invention will be useful where the sequence of a gene or an expressed sequence of interest has been elucidated. This sequence can be used as a basis for screening a collection to identify those paired samples which represent an animal carrying a mutant copy of the sequence. The sample of reproductive material from the paired samples can then be used to produce an animal carrying a mutant copy of the gene of interest.

Although the samples must be “paired” this does not mean, for instance, that the samples must be stored together or even in close proximity. Nor does it mean that there must only be two samples per mutagenized animal. Rather, it means that there must be some way of identifying the sample of reproductive material which corresponds to a given sample of genetic screening material, and vice versa. Or that there must be some way of identifying the animal and its corresponding sample of genetic material. Whether this is achieved by keeping (housing) and/or storing the samples in pairs, for instance, or by keeping and/or storing samples individually in conjunction with an index which can be used to identify the corresponding samples, is a matter of choice and convenience. A mutagenized animal might be represented by more than two different sources of sample (eg., the animal itself, or a tissue thereof such as its spleen, its liver, its brain, and its gametes), each of which might be stored in aliquots, but these are still “paired” according to the invention.

Furthermore, it might be preferred to pool individual samples. For instance, the samples of genetic screening material of more than one mutagenized animal might be stored together, but to retain “pairing” it must be possible to identify the samples of reproductive material which correspond to the pooled material. That is, there must exist a reference collection of mutated reproductive material which is not pooled.

According to a further aspect of the invention, there is provided a collection of samples of reproductive material from a plurality of mutagenized animals, wherein each reproductive material sample is paired with a sample of genetic screening material from the same animal. Similarly, there is provided a collection of samples of genetic screening material from a plurality of mutagenized animals, wherein each sample of genetic screening material is paired with a sample of reproductive material from the same animal.

According to a further aspect of the invention, there is provided a method for identifying samples in a collection according to the invention which carry a mutation in a gene of interest, comprising the step of screening the samples of genetic screening material in the collection to identify those samples which carry the mutation.

Depending on the method used, the genetic screening material is preferably screened using a probe comprising the wild-type sequence of the gene of interest.

The preferred screening method is SSCP, which has proven useful for detection of mutations and polymorphisms.

According to a further aspect of the invention, there is provided a method for selecting from a collection according to the invention a paired sample which carries a mutation in a gene of interest, comprising the steps of: screening and identifying a sample of genetic screening material as described above; and selecting the paired sample which comprises said identified sample.

According to a further aspect of the invention, there is provided a method for producing an animal carrying a mutation in a gene of interest, comprising the steps of: selecting a paired sample as described above; and using the sample of reproductive material from said paired sample to produce progeny carrying said mutation.

According to a further aspect of the invention, there is provided a method for identifying a phenotype associated with a mutation in a gene of interest, comprising the steps of: producing progeny as described above; and examining the progeny for aberrant phenotypes.

According to a further aspect of the invention, there is provided a process for generating a non-human animal model for a human disease caused by a defect in a gene of interest, comprising the steps of: screening a collection according to the invention for a paired sample carrying a mutation in the animal homologue of the gene of interest; using the sample of reproductive material in said paired sample to produce an animal carrying the mutation.

### DESCRIPTION OF THE DRAWINGS

FIG. 1 is a schematic illustration of generation of a paired sample library from mice according to the invention. Male mice are treated via intraperitoneal injection with the DNA mutagen ethylnitrosourea (ENU). Sperm from these mice contain random mutations, and when mated with a nonmutagenized female, the resulting F1 offspring carry heterozygous mutations in their somatic and germ tissue. Reproductive cells, either sperm or ova, are isolated from each mouse and stored in an array at −196 degrees Celsius. These cells can be used to regenerate a living mouse. In addition, somatic tissue, such as the spleen, is isolated from each mouse, and provides a source of DNA for mutation analysis. These samples are identified and arrayed such that each reproductive sample has a corresponding somatic sample from the same mouse.

FIG. 2 is a schematic illustration of a screening process useful according to the invention for mutations using the test sample from a paired sample collection. DNA samples are pooled (in this example DNA from three different animals containing ENU induced mutations), and this pool is subjected to several separate PCR reactions using fluorescently labelled PCR primers. (In this example, three separate PCR reactions; one with blue, one with green, and one with yellow labelled primers.) Each primer pair amplifies a region of a gene to be tested by the fluorescent single strand conformation polymorphism (FSSCP) assay for mutations. The different colour PCR products from each pooled DNA amplification are combined and run on a single lane of a polyacrylamide gel in an ABI 377 DNA sequencing machine under SSCP conditions. Additional lanes are loaded with PCR products obtained in the same manner, but derived from pools of DNA from different animals. Altered fragment mobilities of a PCR product from mutagenized mice relative to mobilities of the same fragment from nonmutagenized mice indicate a mutation in the region being tested. Colour of the product identifies which PCR product contains the mutation, hence which region of DNA contains the mutation. As the PCR was originally performed on a pool of animals, the fSSCP procedure is repeated for each animal in the pool individually. This identifies which animal contains the mutation. Sequencing of this region of DNA precisely identifies the mutation. The corresponding reproductive material portion of the paired sample is then used to generate a mouse carrying the same mutation as that identified by screening the test sample.

FIG. 3 is a schematic illustration of a screening process according to the invention using paired samples to regenerate a mouse from reproductive cells corresponding to a tested DNA sample. The test sample component (in this case DNA) of a paired sample collection is screened for mutations. Upon identification of a sample containing a mutation, the paired regenerative sample (in this case sperm) is retrieved from the corresponding paired sample array and used to generate a mouse carrying the same mutation as that identified by screening the test sample.

FIG. 4 illustrates a mouse breeding scheme according to the invention. The steps are indicated for breeding mice containing mutations to study phenotypes associated with mutations.

### DESCRIPTION

The invention encompasses collections of paired mutations, wherein the mutations within any one pair are identical. The collections may be provided in the form of a collection of pairs of samples of genetic screening material (the first pair member) and reproductive material (the second pair member), or they may be provided in the form of pairs of mutated genomes in the form of an animal carrying one or more induced mutations (the first pair member) and the animal's sample genomic material (the second pair member).

The contents of publications disclosed herein are incorporated by reference in their entirety.

Methods of Inducing Mutations/Mutation Frequency

Many suitable methods for inducing mutations are known in the art. These include chemical mutagenesis, radiation, and retroviral or transposon insertion. Preferably the mutagenesis method is applicable to induce mutations in the genomic material of a living animal (i.e., in vivo), and induces point mutations; insertional mutations are preferably not employed. Preferred methods of chemical mutagenesis involve exposure to alkylating agents such as ethyl- or methyl-nitrosourea (ENU or MNU) [eg. Russell et al. (1983) Environ Mutagen 5, 498; Russell et al. (1984) Environ Mutagen 6, 390]. The most suitable method may depend on the animal in question, but the particular choice is routine [methods for the zebrafish, for instance, are described in Rossant et al. (1992) Of fin and fur: mutational analysis of vertebrate embryonic development. Genes Dev 6,1, and for the mouse in Rinchik E (1991) Chemical mutagenesis and fine-structure functional analysis of the mouse genome. TIG 7, 15-22]. The particular method used might also depend on the target for mutagenesis, such as germline or somatic cells.

Types of DNA Mutations

Mutations in DNA may be (a) large lesion mutations (on the order of kilobases), such as chromosomal breaks or rearrangements; (b) small lesion mutations, such as cytogenetically visible deletions within a chromosome; and (c) small alterations, such as point mutations, insertions and small deletions (on the order of several-tens of bases). Any type of mutation may be used in generating a collection of paired mutations according to the invention, although insertional mutations on the order of several hundred base pairs or larger are not preferred.

The invention is most useful for analyzing the latter category of mutations, i.e., point mutations, insertions and small deletions (2-3 nucleotides, or less than about 200 nucleotides), and therefore it is preferred that the mutagenesis technique used to induce mutations to generate a collection of paired mutations according to the invention induce these types of mutations.

Selection of Mutagenesis Technique

The selection of a mutagenesis technique useful in generating collections according to the invention is dependent upon several factors. Some mutagens cause a wide spectrum of mutation types at a fixed condition(s). Some mutagens cause different types of mutations depending upon the mutagen dosage, mode of delivery, and the developmental stage at which the mutagen is administered to the animal. In addition, a mutagen may induce mutations at different frequencies depending upon the dosage regimen, mode of delivery, and the developmental stage of the animal or cell upon mutagen administration, all parameters of which are disclosed in the prior art for different mutagens or mutagenesis techniques. In addition, a defect in a gene which in wild-type form prevents mutations from occurring or repairs mutations may result in the failure to repair DNA mutations and thus provide a mutagenized genome for generating a collection of paired mutations according to the invention. Finally, the mutation rate from tissue to tissue will vary.

A mutagen or method of inducing mutations is considered useful according to the invention which provides the highest number of mutations per genome which does not kill the mutated animal.

Therefore, the following guidelines are important for selection of a mutagenesis technique or a mutagen for use according to the invention. First, the number of potentially mutant animals which are generated for screening must be technically feasible. Second, the technique used to screen the generated animals for mutations in a given gene or genes must be technically feasible. Third, the type of mutation induced in a gene of interest must leave the gene intact in the genome to the extent that it is detectable as described herein, with small deletions/insertions/substitutions, such as single base pair to several base pairs, being preferred. With these considerations in mind, it is possible to produce a collection of animals which have been mutagenized at a high frequency or at a low frequency.

Those mutagens or mutagenesis techniques which result in mutations which occur within a gene, i.e., a region of DNA from which RNA is transcribed, or within the regulatory elements controlling expression of the gene are most useful according to the invention. Chemical mutagens which result in such mutations include but are not limited to mutagens which are alkylating agents which cause single nucleotide changes.

Therefore, to generate a collection of paired mutations according to the invention, mutations are induced in an animal at a high enough frequency such that the number of animals needed to provide a mutation in a gene of interest is not prohibitive. For example, it is particularly useful according to the invention to induce mutations at a high frequency in order to decrease the number of animals that need to be screened to identify a mutation. ENU mutagenesis is particularly useful in generating a collection of paired mutations according to the invention because, in the offspring of ENU mutagenized male mice, a mutation in any given gene will occur at a frequency of approximately 1 per 1000 mice. Thus, approximately 1000 mice are screened in order to detect a mutation in a particular gene. Although the ratio of 111000 has been calculated in the prior art based on phenotypic assays, it is the only way of assessing the relative mutational frequencies of mutagens or mutagenesis techniques useful according to the invention, as direct DNA analysis of the frequencies of mutations induced by a given mutagen or mutagenesis technique has not been performed. Because phenotypic mutation frequencies are based on DNA mutations which alter or destroy the function of a protein such that it causes a phenotypic change, the number of changes in the DNA of these mice in a given gene will be higher than 1/1000 due to “silent” mutations, i.e., which do not result in a phenotypic change. The same type of mutation frequency is obtained using other chemical mutagens, such as MNU, PRC, and MMS. Additional mutagens which may be considered equally useful according to the invention include chlorambucil and melphalan, and those listed below and in Table 1.

Although the mouse is specifically embodied herein as a representative animal that is useful in generating a collection according to the invention, the invention is not limited to the use of mice. For example, other rodents such as a rat or hamster also provide representative animal models. Non-rodent animals are equally appropriate, for example, animals such as insects, nematodes, or fish, such as the zebrafish or medaka fish.

Lower animals are also useful according to the invention, such as mutagenized insects, e.g., Drosophila. EMS mutagenesis has been performed extensively on Drosophila melanogaster (Ashburner, 1989, Drosophila, A Laboratory Handbook, Cold Spring Harbor Laboratory, Cold Spring Harbor, N.Y.; Grell et al., 1981, Drosophila. Environ. Mutagen. 3:381; Ondrej, 1971, Drosophila melanogaster Mut. Res. 12:159). Non-insect primitive animals such as the round worm, Caenorhabditis elegans, may also be used according to the invention. EMS has been used to mutagenize C. Elegans (Wood, 1988, The Nematode C. Elegans, Cold Spring Harbor Laboratory, Cold Spring Harbor, N.Y.).

Non-mammalian animals, such as fish, nematodes, and insects, are particularly useful for generating a collection of paired mutations according to the invention in providing a collection of pairs of mutations for genes which are suspected to play a role in early development of the animal, e.g., in embryonic development, such as pattern-forming genes, limb-forming genes, or organ-forming genes.

From the above description, it is evident that, for generating a collection of paired mutations according to the invention, mutations also may be induced in an animal at a lower frequency (for example, where a mutagen is used having a lower mutation-induction frequency), provided a higher number of animals or tissue samples from animals are screened for a mutation in a gene of interest. The number of animals tested is generally limited by the following: the number of mutant animals that are generated, and the number of animals that are screened. It may be possible to generate and screen a sufficient number of animals to detect even an exceedingly low frequency of mutation. Although screening for mutations which occur at a given frequency may be labor-intensive, a screening procedure must be employed which is feasible.

The invention therefore contemplates the use of any type of mutagenesis technique, including chemical mutagenesis, radiation mutagenesis, and to mutagenesis techniques which are based on molecular biology, such as introduction into an animal of a gene encoding a defective DNA repair enzyme, retroviral insertion mutagenesis and promoter- and gene-trapping mutagenesis, as described below.

The invention is particularly useful where the mutagenesis results in germline mutations, i.e., which are passed onto offspring which are tested for mutations, and therefore relates to mutations which are induced in the germline of a parent animal.

In a preferred aspect of the invention, a mutagenesis technique is employed which confers a mutation rate in the range of 1 mutation per 500 genes-1 mutation per 10,000 genes, or 1 mutation per gene per 100 animals-1 mutation per gene per 10,000 animals, optimally at least I mutation per 1000 genes, or 1 mutation per gene per 1000 animals. It is desired according to the invention that the mutation frequency possess an upper limit that is below the frequency of inducing a dominant lethal mutation in every animal.

Chemical Mutagenesis and Mutagens.

Chemical mutagens are classifiable by chemical properties, e.g., alkylating agents, cross-linking agents, etc. The following chemical mutagens are useful for generating a collection of paired mutations according to the invention.

The following four mutagens are particularly useful for mutagenesis of male germ cells:

N-ethyl-N-nitrosourea (ENU)

N-methyl-N-nitrosourea (MNU)

procarbazine hydrochloride

chlorambucil

Other chemical mutagens which are useful are as follows:

cyclophosphamide

methyl methanesulfonate (MMS)

ethyl methanesulfonate (EMS)

diethyl sulfate

acrylamide monomer

triethylene melamin (TEM)

melphalan

nitrogen mustard

vincristine

dimethylnitrosamine

N-methyl-N′-nitro-Nitrosoguanidine (MNG)

7,12 dimethylbenz(a)anthracene (DMBA)

ethylene oxide

hexamethylphosphoramide

bisulfan

TABLE 1^{(I)}
Specific-locus mutation rates induced by
chemicals that are mutagenic in post-cell stages of spermatogenesis

| | | Period of | | | Induced mutation rate\mathring{1} | | |
|---|---|---|---|---|---|---|---|
| | | maximum | Exposure2 | | per locus | | Lethal\mathring{3}/tested |
| Chemical | Ref. | effect days\mathring{4} | mg/kg | mol × 10⁻⁵ | × 10⁻⁵ | per mol | mutations |
| Cp | A | 1-14 | 120 | 46.0 | 24.3 | 0.5 | 3/5 |
| MeMs | B | 5-12 | 40 | 36.3 | 24.0 | 0.7 | 10/14 |
| EtMs | B | 5-12 | 175 | 141.0 | 20.9 | 0.1 | 0/1 |
| Et₂SO₄ | C | 5-12 | 200 | 129.7 | 11.2 | 0.1 | 4/4 |
| AA | I | 8-14 | 250 | 351.6 | 18.2 | 0.1 | 1/2 |
| TEM | D | 8-21 | 0.2 | 0.1 | 33.9 | 346.2 | 7/8 |
| Chl | I | 15-21 | 10 | 3.3 | 127.3 | 38.7 | 1/4 |
| Pre | E,F | \mathring{8} | 600 | 232.8 | 21.6 | 0.1 | 1/4 |
| ENU | G | 32-38 | 50 | 42.7 | 10.6 | 0.2 | 0/5 |
| MNU | H | 36-42 | 75 | 72.7 | 90.2 | 1.2 | 0/17 |

Cp, cyclophosphamide; MeMS, methyl methanesulforate; EtMs, ethyl methanesulforate; Et₂SO₄, diethyl sulfate; AA, acrylamide monomer; TEM, triethylene melamine; Chl, chlorambucil; Prc, phocarbazine hydrochloride; ENU, N-ethyl-N-nitrosourea; MNU, N-methyl N-nitrosourea.
\mathring{1}Expressed per kg of body weight. When results for more than one exposure level of a chemical were available, we list the one that the investigator(s) found most suitable for generating mutation-rate data.
\mathring{2}Experimental minus historical control, 43/801, 406, for period of maximum response.
\mathring{3}Letha1s excluded. For chlorambucil, the number includes mutations for which there is genetic, cytogenetic, and/or molecular evidence for deletion.
\mathring{4}Postexposure.
\mathring{8}Experiment did not involve sequential matings.
References:
A. Ehling, U. H. & Neuhauser-Klaus, A. (1988) Mutat. Res. 199, 21-30.
B. Ehling, U. H. & Neuhauser-Klaus, A. (1984) in Problems of Threshold in Chemical Mutagenesis, eds. Tazima, Y., Kondo, & Kuroda, Y. (Environ, Mutagen. Soc. Jpn., Mishima, Japan), pp. 15-25.
C. Ehling, U. H. & Neuhauser-Klaus, A. (1979) Mutat. Res. 199, 191-198.
D. Cattanech, B. M. (1967) Mutat. Res. 4, 73-82.
E. Ehling, U. H. & Neuhauser-Klaus, A. (1979) Mutat. Res. 59, 245-256.
F. Kratochvilova, J., Pavor, J. & Neuhauser-Klaus, A. (1988) Mutat. Res., 198, 295-301.
G. Russell, W. L. & Hunsicker, P. R. (1983) Environ. Mutagen. 5,498 (abstr.).
H. Russell, W. L. & Hunsicker, P. R. (1984) Environ. Mutagen. 6,390 (abstr.).
I. Russell et al., 1989, Proc. Nat. Aca. Sci. 86:3704

ENU Mutagenesis in Particular

One particularly useful mutagen for generating a collection of paired mutations according to the invention is the chemical mutagen ethylnitrosourea (ENU). ENU may be used to induce genomic mutations in any animal, including but not limited to lower animals such as insects and worms, as well as higher animals such as vertebrates, e.g., mammals, e.g., rodents such as mice and rats, hamsters, primates, and zebra fish, cows, sheep, pigs, and dogs. Mutagenesis and DNA mutation screen also may be applied to other animals which are used as model systems for human disease. Rats are a good candidate for practical reasons, i.e., since mouse-based animal facilities are able to breed and maintain rats. The inventive methods are easily applicable to the rat and provides a method for producing and identifying mutations in specific rat genes.

Described below is the applicability of ENU mutagenesis of mice.

Previous mutagenesis experiments used in excess of 500,000 mice for which mutagenesis was induced by the chemical mutagen ENU. The genes involved were assayed indirectly by observation of phenotypic changes in the mice.

ENU is believed to produce mutations at random throughout the genome, and the frequency of mutations, determined for numerous genes, is in the range of 0.5-1.5 mutant mice per 1000 mice, for any given gene screened. In the past, the presence of mutations could only be inferred on the basis of a phenotype in the mutated mice. Most of these mutations do not produce an obvious phenotypic change in the heterozygous state and required additional breeding to make the mutations homozygous (F2 and F3 generations) to observe the effect of the mutation.

Mutagenesis and mutant screening for generating a collection of paired mutations according to the invention does not require a previously-determined mutant phenotype, as the F1 generation mouse DNA is analyzed directly for the presence of a mutation in the gene of interest. In 1000 mice, 0.5-1.5 mutations in any gene which will lead to a phenotype may be detected. By screening 10,000 mice, it is possible to identify 5-15 mice, each carrying heterozygous mutations in a target gene. Any number of genes can be screened in these same 10,000 mice. Assuming 100,000 genes in a mammalian genome, then each mutagenized mouse is carrying mutations in one copy of approximately 100 different genes. The additional mutant genes in each mouse are easily removed by breeding. ENU mutagenesis of mice is performed as described herein.

Using ENU mutagenesis, it is expected that the gene of interest will be mutated to produce a phenotype once in 1000 mice. If a given animal genome contains, for example, 100,000 genes, then each ENU mutated animal will contain in its ENU mutated genome one protein-altering mutation in one allele of every 100 genes.

ENU mutagenesis also may be carried out on rats, following a procedure similar, if not identical to ENU mutagenesis of mice.

ENU mutagenesis also may be carried out on zebrafish, as described herein for ENU mutagenesis of mice.

Radiation Mutagenesis.

In general, Xrays, gamma rays, neutrons, etc., cause DNA breakage. Cellular repair mechanisms of DNA breaks result in regions of DNA which contain large lesions, including rearrangements and deletions. Although the presence of other types of mutations may be preferred according to the invention, radiation induced mutations, which tend to be larger in that they encompass more bases, are also encompassed by the invention.

UV light-induced mutations are largely single nucleotide alterations. However, because UV light does not penetrate an animal, it is used for inducing mutations in cells in culture or on exposed tissues of an animal, e.g., eyes, skin. UV mutagenesis is useful according to the invention for mutagenizing ES cells.

In addition to chemical or radiation induced mutations, mutations may be induced in an animal using any one of a number of mutagenesis techniques.

Mutation Frequency

The frequency of mutation chosen may depend on the number of mutagenized genomes represented in the collection and the coverage of the genome which is desired. If the collection contains a small number of paired samples, in order to cover the whole genome such that a mutant copy of every gene is represented in the collection it is necessary to use a relatively high mutation frequency. If the collection is very large, a low mutation frequency is sufficient. There is, of course, an upper limit to the frequency of mutation which can be induced without resulting in death of the animal, and a lower limit below which the genome will not be fully covered.

A low number of mutations per animal is desirable because possible complications associated with interactions between mutations are reduced and also because it reduces the need for breeding out additional mutations in genes not of interest. On the other hand, as the size of the collection increases, the effort involved in screening the collection also rises.

The mutation frequency is preferably such that, on average, about 10 functionally mutant copies of each gene are present per 10000 samples. At the DNA level the frequency might be more than 10 mutant copies per 10000 samples, but many of these mutations will not affect the function of the gene. The range of these “silent” mutations is diverse, but depending on the gene in question they might be mutations in non-coding regions, point mutations which do not alter the function of a codon (eg CCU to CCG, or CGG to AGG), and mutations which alter a codon but which ordinarily do not affect the final protein function, such as conservative amino acid substitutions (eg. CUU Leu to AUU Ile). The functional mutants in the collection preferably carry mutations distributed across the gene of interest, that is to say throughout its coding and regulatory regions.

ENU mutagenesis is particularly suitable for mouse mutagenesis since, in the offspring of mutagenized male mice, a functionally aberrant copy of any given gene will occur at a frequency of approximately 1 per 1000 mice. Thus a mutant copy of each gene will be represented in the collection on average once every 1000 paired samples. Mutation frequencies for a variety of suitable mutagens in mice have been reviewed in Russell LB et al. (1989) Chlorambucil effectively induces deletion mutations in mouse germ cells. PNAS USA 86, 3704-3708 (See especially Table 2).

A suitable mutagenized population of animals might also be produced by breeding from animals having mutant housekeeping genes such as those coding for DNA repair enzymes or proof-reading enzymes [eg. McWhir et al. (1993) Mice with DNA repair gene (ERCC-1) deficiency gave elevated levels of p53, liver nuclear abnormalities and die before weaning. Nature Genetics 5, 217-224].

Genetic Screening Material According to the Invention

Genetic screening material according to the invention may be any material suitable for genetic analysis, including intact cells and cellular extracts from which DNA or RNA may be isolated for use in assays, and including purified DNA or RNA. The DNA may be in the form of genomic DNA or cDNA. The genetic screening material described in detail herein below is DNA purified from a diploid cell from an animal.

Preservation of Genetic Screening Material

The preservation of genetic screening material without affecting the suitability of the is material for genetic analysis is routine. For example, freezing can be used but, depending on the nature of the sample, other suitable procedures include cryopreservation and lyophilization.

Reproductive Material According to the Invention

Reproductive material according to the invention in each of the paired samples may be any material which can be used to generate progeny of the parent animal, for example, gametes (i.e. spermatozoa or ova). Rather than having gamete samples in the collection, however, it may be preferable to use gametogenic stem cells from which gametes of the parent animal can be produced. In some cases, the reproductive material may be an embryo generated using the parent animal's gametes, or embryonic stem cells from such an embryo. The reproductive material might also be cells suitable for nuclear transfer for cloning the parent animal [Wilmut et al.(1997) Viable offspring derived from fetal and adult mammalian cells. Nature 385, 810-813], or nuclei from such cells.

Preservation of Reproductive Material

The preservation of suitable reproductive material is also known. For instance, gamete preservation without affecting viability has been reported for many species. Suitable procedures have long been used for preserving spermatozoa, for example for artificial insemination in mammals such as cattle, horses and humans, and similar approaches for murine gametes has been described [Nakagata (1995) Studies on cryopreservation of embryos and gametes in mice. Exp. Anim. 44, 1-8].

Preparation and Storage of Tissue Samples, Cell Samples, DNA Samples

A DNA sample for analysis according to the invention may be prepared from any tissue or cell line, and preparative procedures are well-known in the art. Tail tissue is prepared as follows.

A DNA sample for analysis according to the invention may be prepared from any tissue or cell line, and preparative procedures are well-known in the art. The preparation of genomic DNA from tissue is performed as follows. Approximately 0.1 μg of the tissue is removed from a 10-day old mouse, and placed in 500 μl TB buffer (50 mM Tris-HCl, pH 8.0, 100 mM NaCl, 1% SDS, 600 μg/ml proteinase K) and incubated overnight at 55° C. The sample is then extracted with 500 μl 1:1 (w/w) phenol/chloroform and precipitated with two volumes ethanol. The DNA pellet is then resuspended in 500 μl H₂O.

Approximately ⅓ of the tail is removed from a 10-day old mouse, and placed in 500 μl TB buffer (50 mM Tris-HCl, pH 8.0, 100 mM NaCl, 1% SDS, 600 μg/ml proteinase K) and incubated overnight at 55° C. The preparation of genomic DNA from tail tissue is performed as follows. The sample is extracted with 500 μl 1:1 (w/w) phenol/chloroform and precipitated with two volumes ethanol. The DNA pellet is then resuspended in 500 μl H₂O. The DNA pellet or resuspended DNA may be stored frozen (⁻20 or ⁻70° C.).

cDNA samples also may be prepared according to the invention, i.e., DNA that is complementary to RNA such as mRNA. The preparation of cDNA is well-known and well-documented in the prior art. cDNA is stored under the same conditions as DNA.

Tissues which are useful according to the invention include but are not limited to blood cells, brain, gonad, liver, heart, kidney, adrenal, spleen, and muscle. Tissue samples are provided by obtaining a tissue sample from an animal, freezing the tissue sample as described herein, and DNA may then be isolated from the frozen tissue sample, or an aliquot thereof, as described herein. All tissues may be stored frozen at ⁻20-⁻70° C. in separate tubes identifiable as a member of a paired mate. Similarly, cell samples from such tissues are provided according to routine methods. Tissue samples, cell samples, and DNA samples may be stored frozen (⁻20-⁻70° C.), or as desired, and are typically thawed on ice prior to DNA extraction.

Effect of Breeding Mutagenized Animals

If a mutagenized animal is mated with a non-mutagenized animal, the resulting F1 generation will be heterozygous for the mutations. Although samples of reproductive material for the collection can be prepared from this F1 generation, once the cell undergoes meiosis only half of these F1 gametes will carry any particular mutation present in the parent animal and so not all of them are capable of producing progeny carrying that mutation. In any given aliquot of a gamete sample, however, the mutation will be represented and so each aliquot will contain reproductive material suitable for generating progeny carrying that mutation.

If a gamete carrying a mutation is crossed with a non-mutagenized gamete, the resulting progeny will be heterozygotic for that mutation. If the mutation is recessive, this will not result in a mutant phenotype. In order to produce animals homozygous for the mutation of interest, further breeding may be necessary. This breeding is not required to carry out screening according to the invention, however, since the screening is independent of phenotypic observation; rather it is necessary in order to observe the phenotype caused by a recessive mutation.

Pooling of Samples According to the Invention

It may be desirable according to the invention to pool samples. As used herein, “pooling” refers to the pooling of a given number of first members of the pair with other first members, or pooling of a given number of second members of the pair with other second members. For instance, the samples of genetic screening material from more than one mutagenized animal might be stored together, but to retain “pairing” it must be possible to identify the samples of reproductive material which correspond to the pooled material. The samples may be individually provided, or they may be provided in pools (e.g., 2-50 individual samples, preferably 2-10, more preferably 2-5 individual samples), as long as individual paired samples are also maintained. For instance, if samples of genetic screening material from ten animals are stored together, these samples can be screened together, and if a mutation of interest is identified in the pooled samples then the corresponding samples of reproductive material must be identified. Obviously, where pooling is used it is necessary to deconvolute the pooling step in order to identify the contributions of the individual samples. For instance, the progeny produced from pooled gametes will represent more than one mutagenized parent animal and it will be necessary to screen the progeny to determine which carry the mutation of interest. Therefore pooling concentrates the samples for screening purposes, but dilutes the mutation of interest for reproductive purposes. However, it must always be possible to identify the sample of reproductive material which corresponds to a sample of genetic screening material, and vice versa, even if pooling has been used.

Collection of Mutagenized Animals According to the Invention

A collection of animals according to the invention can include an animal of any type, for example a vertebrate such as a mammal or a fish. Mammals useful according to the invention include primates, rodents (mice and rats), lagomorphs, guinea pigs, horses, sheep, cattle, goats, pigs, cats, and dogs. Preferred fish are zebrafish and medaka fish.

Where the collection of animals is a collection of mutagenized rodents such as mice, the following protocol is used to house and perpetuate the collection. The animals are housed in a mouse facility which conforms to government regulations for animal care. There are several veterinarians who supervise and monitor the animal welfare. C3H male mice are injected interperitoneally with ENU. About 150 males are injected every 3 weeks to provide breeding stock. They are mated with either one or two untreated females in a cage (a plastic box with wire lid). Every couple of days the males are put in with new females, each of which will have 5-6 offspring (F1). The females are pregnant for 3 weeks (21 days) and after birth the babies are kept with their mothers for 3 weeks, at which time they are weaned, and a little clip of tail is taken before the babies are transferred into single sex cages (boxes), each housing 6-7 mice. The tail clip is taken at this time because mice of that age do not react to the clip. At later ages they do react, and would need anesthetic, while at earlier ages the tail is smaller, yielding less DNA. Also it is convenient, as at weaning the mice are given a unique identifying number and are being handled anyway for transfer to another cage. A room holds about 300 cages, with roughly 1750 mice per room. Six rooms is sufficient to house a given collection of mice. The population of mice is kept at ˜10,000. Once at 10,000 population, 2,000 new arrive each month and the 2,000 oldest (aged 5 months) depart. This is done because virgin female mice will not mate after a few months, although we can always obtain eggs from them and use IVF to recover. Male mice also lose interest in mating after approximately 9-12 months.

Where the collection of animals according to the invention is a collection of fish, a representative fish is the zebrafish. The zebrafish is a striped 2-inch long fish from the Ganges River. The zebrafish has been used as a genetic system and conditions for gamma-ray mutagenesis and screening are well-established (Chakrabarti et al., 1983, Brachydonio Genetics 103:109; Walker and Streisinger, 1983, Genetics 103:125). The advantages of zebrafish over the mouse for genetic analysis is its small size, the ability to house a large number of animals cheaply, and the large number of embryos produced from one female (usually a few hundred but as many as 1000 eggs). The time from fertilization to gastrulation is only about 5 hours at 28 C; somites form between 10-20 hours; and by 24 hours postfertilization, a recognizable animal with rudimentary eyes and brain has formed. Thus, the early development of this vertebrate takes only about as long as a phage plaque assay. Rossant et al., 1992, Genes & Development 6:1, describe mutational strategies for mutagenesis of zebrafish, including ENU mutagenesis.

Briefly, a three-generation cross in which F2 females, heterozygous for a number of induced mutations, are backcrossed to their father and mated to their brothers to reveal homozygous mutant phenotypes. A locus-specific mutation frequency of 1/1000 gametes scored is achievable in zebrafish using ENU mutagenesis. Therefore, one would need to screen at least 3,000 mutagenized gametes to approach saturation mutagenesis, and fewer than 2,000 gametes, i.e., on the order of about 1,000 gametes to perform mutational analysis according to the invention. ENU and EMS mutagenesis has been used to induce mutations in isolated sperm from zebrafish (Halpern et al., 1993, Cell 75:1; and solnica-Knezel et al., 1994, Genetics 136:1401). The small teleost fish Medaka has also been subjected to ENU mutagenesis (Shiva et al., 1991, PNAS 88:2545), and also is encompassed within the invention. Zebrafish have been used in large-scale mutagenesis to search for genes controlling development in vertebrates (Mullins et al., 1994, Curr. Biol. 4:189).

Screening Methods

The screening method can be any technique for detecting sequence differences. Suitable methods include nucleotide sequencing, single stranded conformation polymorphism (SSCP) [Orita et al. (1989) Detection of polymorphisms of human DNA by gel electrophoresis as single-strand conformation polymorphisms. PNAS USA 86, 2766-2770], denaturing gradient gel electrophoresis, sequencing by hybridization to an oligonucleotide array [Chee M et al. (1996) Accessing genetic information with highdensity DNA arrays. Science 274, 610-614], chemical cleavage of mismatches, RNase cleavage, and mismatch recognition by DNA repair enzymes such as MutS [Eng et al. (1997) Genetic testing: the problems and the promise. Nature Biotech 15, 422-426, (see especially “Best Bets for genetic testing: mutation scanning methods”)].

The preferred screening method is SSCP, which has proven useful for detection of multiple mutations and polymorphisms [eg. Glavac et al. (1993) Optimization of the single-strand conformation polymorphism (SSCP) technique for detection of point mutations. Hum Mut 2, 404414]. SSCP sensitivity varies with the length of sequence being analysed and the optimal size appears to be 150-300 nucleotides. Preferably the screening method is fluorescence SSCP (fSSCP), which can be analysed using an ABI™ fluorescent DNA sequencing machine.

Where the screening method is optimal for sequences shorter than the complete gene of interest, it may be necessary to screen the gene in a number of segments which span its whole length. Where genomic DNA is being screened, it may be preferable to screen the gene in segments corresponding to the exons.

Usually it will be necessary to amplify the gene of interest, or segment thereof, prior to detecting any sequence differences. Preferably this amplification is by PCR, utilizing primers unique to the sequence to be tested. Where the screening method utilizes fluorescence or radioactivity, the PCR products should be suitably labelled.

There are three general approaches for screening the samples. Firstly, samples are screened in order to provide mutation detection for a single gene using a probe which is unique to that gene. Secondly, samples are screened using a mixture of unique probes spanning a gene, providing simultaneous mutation detection across the gene examined, for instance where the complete gene is longer than the sensible limit for amplification or for mutation detection. Thirdly, samples are screened using a mixture of probes for different genes, providing simultaneous mutation detection for several different genes. Any of these three approaches can be combined with the pooling of different samples so that a number of samples can be screened simultaneously (multiplexing).

Preferably the screening method is optimized to allow simultaneous screening for a plurality of different mutations and/or simultaneous screening of a large number of samples. Such optimization accelerates the screening of the collection.

Where the gene is screened in segments, for instance, it is preferable to screen a plurality of those segments for mutations simultaneously. This will involve the use of a plurality of nucleic acid probes collectively spanning the gene of interest, each probe being unique. These probes will usually be produced by amplifying the segments using different sets of amplimers for each segment.

Once the presence of a mutation has been detected, the actual sequence mutation can, if desired, be determined by sequencing nucleic acid derived from the particular mutant gamete sample or from an animal generated therefrom.

It will be understood that the invention is described by way of example only and modifications may be made while remaining within the scope and spirit of the invention.

### Collections According to the Invention

In Examples 1 and 2, the preparation of three different collections of paired mutations according to the invention is described: a) a set of mutagenized animals paired with a corresponding set of gamete tissue or DNA, wherein the animals may be one of reproductive material or genetic screening material and the gamete tissue is the other; b) a set of F1 generation (derived from the mating of mutagenized animals with nonmutagenized animals) animals paired with a corresponding set of tissue (somatic or gamete tissue) or DNA thereof, wherein the animals may be one of reproductive material or genetic screening material and the tissue is the other; and c) a set of F1 somatic tissue (or DNA thereof) which is genetic screening material paired with a set of gametes (or DNA thereof) which is reproductive material. These collections according to the invention are prepared as follows in mice (Example 1) and rabbits (Example 2).

### EXAMPLE 1

Producing a Collection of Paired Mutations according to the Invention in Mice

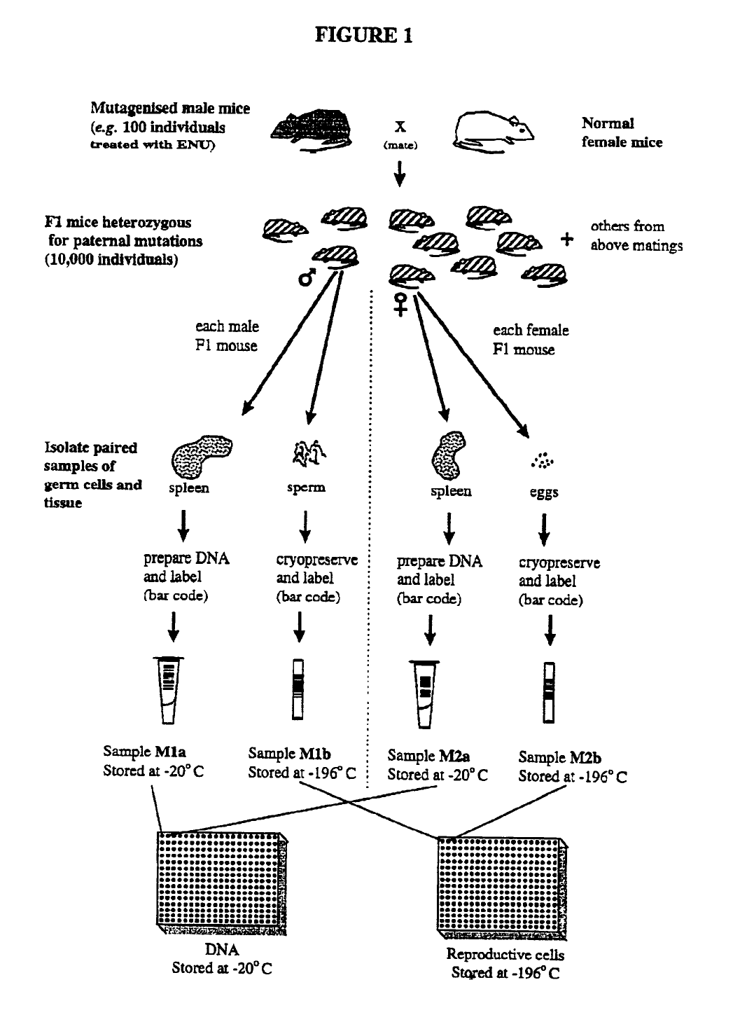

Mutations in the DNA of the premeiotic spermatogonia of male mice can be induced with the DNA alkylating agent ethylnitrosourea (ENU). Typically, 3 separate doses of 100 mg/kg body weight ENU are injected interperitoneally, with each injection separated by a one week interval. The animals undergo a period of sterility (usually 8-14 weeks), after which they can be mated to nonmutagenized females to produce offspring (the F1 generation) which carry heterozygous mutations in their genome (FIG. 1).

A collection according to the invention can be prepared at this post-mutagenesis stage, i.e., at which the mice are again fertile after mutagenesis. Because of the nature of the invention with respect to paired mutations, wherein the pairing refers to mutations which are identical in position and nature in a given genome, a pair may include the mutagenized males (prior to mating to produce the F1 generation) and only its corresponding reproductive tissue (i.e., gametes). That is, the somatic tissue of the mutagenized males is not a member of paired mutations according to the invention because the somatic tissue of a mutagenized male does not contain mutations that are identical to mutations in the gamete tissue.

Each animal carries the same set of heterozygous mutations throughout both somatic and germ tissue. To generate large numbers of offspring for a paired sample set, 300 males are treated with ENU. Approximately 100 of these mice will be permanently sterile; the remaining 200 are permanently mated with 2 females each. This results in the generation of 4000 F1 offspring per month. In this way a set of paired samples comprised of over 30,000 animals can be produced within a year, including the time required to establish the ENU treated breeding males. The process can be scaled up by ENU treatment and breeding of additional male mice.

A collection according to the invention can be made upon production of the F1 generation above (reproductive material) and upon providing a corresponding plurality (1 sample per F1 mouse) of somatic tissue samples or DNA thereof (genetic screening material).

At sexual maturity (about 6 weeks of age), the animals are sacrificed, and gametes (sperm or eggs) and a sample of somatic tissue (for instance spleen), are harvested from each F1 mouse. The samples are given identifiers which link their common origin, and the samples are stored separately. (FIG. 1.)

A collection according to the invention also can be prepared using the F1 somatic tissue or DNA thereof (genetic screening material) which serves as a source to test for DNA mutations, and F1 the gametes (reproductive material) which comprises an immortal source of material to regenerate a mouse corresponding to any of the somatic samples.

### EXAMPLE 2

Producing a Collection of Paired Mutations according to the Invention in Rabbits

For medium size animals, it is not always feasible to generate all of the offspring in a short period of time, due to the resources required for each animal. A paired sample set can be generated over a longer time period for animals such as rabbits.

Multiple, individual male rabbits, e.g. 40, are treated with ENU and mated to nonmutagenized females. Females have 3 to 5 litters per year with a gestation of 1 month and litter size ranges from 4 to 10 and averages 5 to 6. On average a male mated to a single female will produce 24 offspring per year. Using 40 ENU treated males in continuous permanent matings will result in nearly 1000 F1 mutated animals per year. Rabbits reach sexual maturity in 4 to 6 months, at which time the animal is sacrificed, and reproductive cells (sperm or eggs) and a sample of somatic tissue (for instance liver), are harvested from each F1 animal. This process can be continued indefinitely to continually increase the number of samples in the paired sample collection. The samples are given identifiers which link their common origin, and the samples are stored separately. The somatic tissue serves as a source to test for DNA mutations, and the reproductive cells comprise a permanent source of material for regenerating a mouse which corresponds to each and any of the somatic samples.

### EXAMPLE 3

Screening a Collection according to the Invention

The SOX gene family have high sequence similarity between one another in a portion of each SOX gene. Each member contains an approximately 240 bp DNA sequence corresponding to a 80 amino acid segment (called an “HMG box”) that shows 60% or greater amino acid similarity between members. This defines the SOX gene family. Outside of this region the genes are very different.

The HMG box is a DNA binding domain and the SOX genes which have been studied bind to DNA via this region of the protein and are likely modulators of gene expression i.e. transcription factors. There are about 20 different Sox genes known in mouse and a similar number in humans. Several of these genes have been implicated in disease.

SOX9 is a gene which when mutated in humans causes a neonatal lethal chondrodysplasia called campomelic dysplasia (CD). In addition, in CD patients who have XY sex chromosomes and should develop as male, ¾ develop as female (XY sex reversal). Sox-4 when mutated in mouse leads to problems in B cell formation and a cardiac condition which is the same as a cardiac development defect seen in man. It is of interest to discover the function of SOX genes, given their likely role as transcription modulators involved in developmental processes. For some SOX genes, little sequence is known, for others the entire cDNA and genomic structure, plus expression patterns are known.

One gene which has been studies fairly extensively is Sox-3 (both mouse Sox-3 and the human homologue SOX3). A mouse knockout (artificially generated null allele) of Sox-3 has not been reported, but in human there is an individual who has a deletion which removes (minimally) SOX3 and the factor IX (blood clotting) gene, and has mental retardation and haemophilia. SOX3 may be linked to the retardation. Mouse Sox-3 is expressed in the developing central nervous system, adding support to this hypothesis.

A paired sample collection is usefull for identification and generation of mice containing mutations within the Sox-3 gene to study the role of Sox-3 in mental retardation. A collection of paired samples, DNA and mouse reproductive cells, from F1 mice containing ENU induced mutations is generated as in Example 1 and FIG. 1.

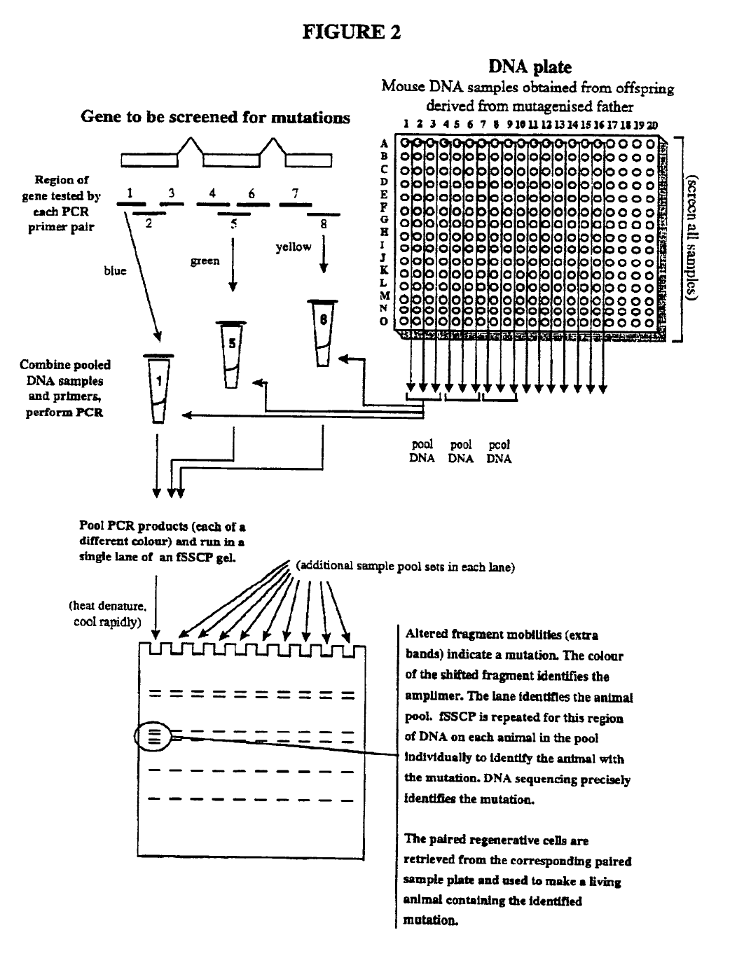

PCR primers are designed to amplify the entire open reading frame of the Sox-2 gene and are used on pooled muse DNA samples as shown in FIG. 2. The DNA component of each sample is screened by FSSCP (FIG. 2), and samples containing mutations predicted to impair or destroy protein function are identified.

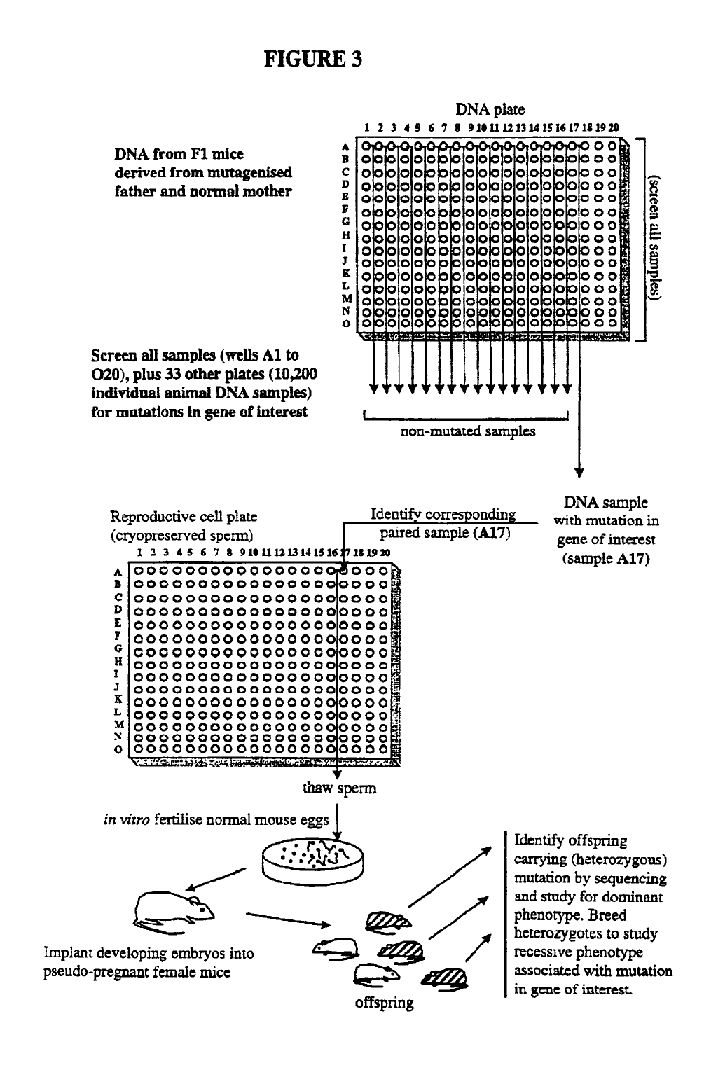

The corresponding cryopreserved spermatozoa or oocytes are then used to generate mice (FIG. 3) according to Nakagata (Exp. Anim. 44:1-8 1995).

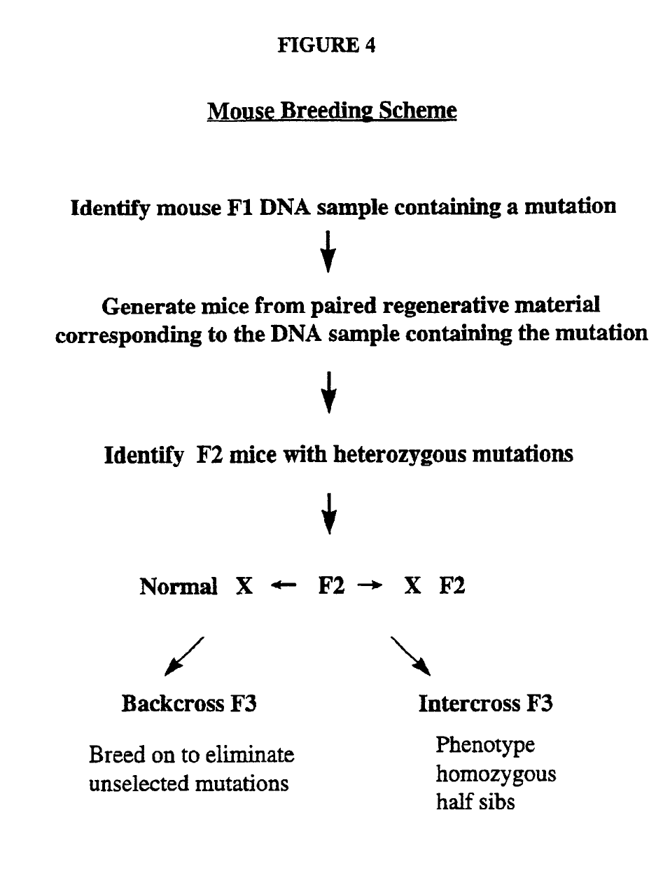

As the animal from which the samples were taken were heterozygous for the mutation, one-half of the mice made from the stored reproductive cells (F2 generation) will contain the identified mutation. The F2 mice containing the mutation can then be interbred to generate offspring which are homozygous for the mutation (FIG. 4). The homozygous mutant animals are examined for a phenotype related to retardation, such as neural defects and impaired learning, as a means to study the function of the Sox-3 gene. The initial ENU treatment generates multiple mutations per mouse. In addition to the identified mutation, there exist random “background” mutations. In each animal from which the paired samples were derived there are approximately 100 background mutations. One-half of these mutations are lost at random with each mating to a normal mouse, so at the F3 generation, there are 49 random background mutations. Of these, 6 at random will be homozygous. As each sib and half sib has different background mutations, multiple mice can be examined to insure that the observed phenotype is resulting from the selected mutation (which is homozygous in all of the animals) (FIG. 4).

Further breeding to normal mice continues to segregate away background mutations, and after approximately 8 generations, the background mutations have been removed.

### USE

Since the advent of gene mapping and DNA sequencing, and the initiation of ventures such as the human genome project, a mass of gene sequence information has been produced, and one of the key challenges in modem genetics is the elucidation of functions for these genes [Friedrich (1996) Moving beyond the genome projects. Nature Biotech 14, 1234-1237]. The human genome is thought to contain around 80000 genes, and while the human genome project aims to provide detailed sequence information for all of these genes, in most cases this will give no indication of the function of the gene product.

Typically, workers will start with a known phenotype and try to identify the genotype responsible. Once the genotype and phenotype are correlated, which can be a laborious process, the gene product can be identified and the molecular basis whereby mutations in that gene causes a disease phenotype can be investigated rationally. In other words, the phenotype is usually used as a guide when searching for mutations in genes.

As an alternative, it is possible to start with a mutated gene and then look for associated phenotypes. The effect of mutations can therefore be explored without prior knowledge of the function of the gene.

Collections according to the invention allow the screening of animal populations for mutations without knowledge of the phenotype caused by the mutation. Where a mutation of interest is identified in the collection, an animal carrying that mutation can be produced.

Collections according to the invention are useful in the discovery and characterization of genes of interest, with a view towards the identification and development of therapeutic agents or targets for therapeutic methods. They are also useful for identifying gene defects involved in disease, thus allowing the development of diagnostics.

Screening methods according to the invention are thus useful for identifying biological samples carrying mutations in useful genes and for producing animals carrying mutations in those genes. These animals can be used for phenotypic characterization of the gene and as models of disease.

Identification of those samples in the collection which carry a mutant gene permits the subsequent assessment of phenotypes resulting from the alteration of gene function and provides a model animal for further disease research. These animals can be used to model human disease. The mutant genes identified, or their wild-type counterparts, may be useful targets for medical, therapeutic, or diagnostic applications.

1. A collection of paired samples from a plurality of mutagenized animals, wherein each of the paired samples comprises: a first sample comprising genetic screening material of an animal; and a second sample comprising reproductive material of that same animal.

2. The collection of claim 1 wherein said first sample comprises an animal of said plurality of mutagenized animals.

3. The collection of claim 1 wherein said first sample comprises a somatic tissue of an animal of said plurality of mutagenized animals.

4. The collection of claim 1 wherein said first sample comprises genomic DNA of a somatic tissue of an animal of said plurality of mutagenized animals.

5. The collection of claim 1 wherein said second sample comprises a fertile animal of said plurality of mutagenized animals.

6. The collection of claim 1 wherein said second sample comprises reproductive tissue of an animal of said plurality of mutagenized animals.

7. The collection of claim 1 wherein said second sample comprises gametes of a fertile animal of said plurality of mutagenized animals.

8. The collection of claim 1 wherein said plurality comprising 100-1,000,000 animals.

9. A method of providing the collection of claim 1, comprising the step of obtaining pairs of samples from a plurality of mutagenized animals: a first sample comprising genetic screening material; and a second sample comprising reproductive material.

10. A method of providing the collection of claim 1, comprising the step of providing said plurality of mutagenized animals and a corresponding plurality of samples of tissue, wherein one of said plurality of mutagenized animals and said corresponding plurality of samples of tissue is reproductive material and the other is genetic screening material.

11. The method of claim 9 or 10, further comprising the step of mutagenizing a plurality of a parent animal.

12. The method of claim 11, further comprising the step of mating said mutagenized parent animal to a nonmutagenized mate to produce said plurality of mutagenized animals.

13. The method of claim 11, said step of mutagenizing being performed in vivo.

14. A collection of pairs of mutated genomes comprising a plurality of animals and a corresponding plurality of sample genomic material, wherein a said pair of mutated genomes of said collection comprises an animal having a genome carrying a mutation present at a frequency substantially above the frequency of a spontaneous mutation, and the animal's sample genomic material, wherein one of said animal and said sample genomic material is reproductive material and wherein the other of said animal and said sample genomic material is genetic screening material.

15. The collection of claim 14 wherein said sample genomic material is reproductive material and comprises gametes.

16. The collection of claim 14 wherein said sample genomic material is genetic screening material and comprises somatic cells or DNA thereof.

17. The collection of claim 14 wherein said plurality of animals comprises mutagenized animals and said corresponding plurality of sample genomic material comprises gamete tissue of said mutagenized animals.

18. The collection of claim 14 wherein said plurality of animals comprises a plurality of F1 animals having a mutagenized parent and a nonmutagenized parent and said corresponding plurality of sample genomic material comprises tissue from the F1 animal, wherein said plurality of F1 animals is one of reproductive material and genetic screening material, and said corresponding plurality of sample genomic material is the other.

19. A method of generating a collection of paired mutated genomes as claimed in claim 14, comprising the steps of mutagenizing an animal, and obtaining a corresponding plurality of sample genomic material from said animal, wherein a said pair of mutated genomes comprises (a) an animal of said plurality of animals and (b) a sample genomic material from said plurality of sample genomic material from said animal.

20. The method of claim 19, further comprising the step of breeding said animal with an unmutagenized mate to produce a plurality of F1 offspring, and obtaining a corresponding plurality of sample genomic material from said F1 offspring, wherein a said pair of mutated genomes comprises (a) an F1 offspring of said plurality of F1 offspring and (b) a sample genomic material from said plurality of sample genomic material from said F1 offspring.

21. The method of claim 19 wherein said animal comprises reproductive material and said sample genomic material comprises genetic screening material.

22. The method of claim 19 wherein said animal comprises genetic screening material and said sample genomic material comprise reproductive material.

23. The method of claim 22, said reproductive material being sperm.

24. The method of claim 20 wherein said F1 offspring comprises reproductive material and said sample genomic material comprises genetic screening material.

25. The method of claim 20 wherein said F1 offspring comprises genetic screening material and said sample genomic material comprise reproductive material.

26. The method of claim 25, said reproductive material being sperm.
